(19) United States
(12) Patent Application Publication
(10) Pub. No.: US 2025/0207289 A1
(43) Pub. Date: Jun. 26, 2025

(54) SYSTEMS, METHODS, AND INTERFACES FOR OPTIMIZING COMPOSITIONAL METRICS OF AN E-COAT BATH

(71) Applicant: PPG INDUSTRIES OHIO, INC., Cleveland, OH (US)

(72) Inventors: Kevin Martin Muck, Pittsburgh, PA (US); Serena Michele Day, Oak Park, MI (US); Cory Matthew Thomas, Oakmont, PA (US)

(73) Assignee: PPG INDUSTRIES OHIO, INC., Cleveland, OH (US)

(21) Appl. No.: 18/852,015

(22) PCT Filed: Mar. 28, 2023
(86) PCT No.: PCT/US2023/065025

§ 371 Date: Sep. 27, 2024

Related U.S. Application Data
(60) Provisional application No. 63/324,520, filed on Mar. 28, 2022.

(51) Int. Cl. C25D 13/24; G16C 20/70

(52) U.S. Cl. CPC C25D 13/24; G16C 20/70

(57) ABSTRACT

A computer-implemented method for maintaining key compositional metrics, such as percent solids and/or pigment-to-binder ratio, in an e-coating bath can include receiving sensor data directly from an e-coat bath, wherein the sensor data correlates with changes in compositional metrics. One or more computer algorithms can process the data in real-time to quickly and accurately identify recommended adjustments to the e-coat bath.

### BACKGROUND

### 1. Technical Field

The present disclosure relates to devices, computer-implemented methods, and systems for optimizing key compositional metrics in an electrocoat environment.

### 2. Background and Relevant Art

Modern coatings provide several important functions in industry and society. Coatings can protect a coated material from corrosion, such as rust. Coatings can also provide an aesthetic function by providing a particular color and/or texture to an object. For example, most assets such as automobiles are coated using paints and various other coatings in order to protect the metal body of the automobile from the elements and also to provide aesthetic visual effects.

In some environments, coating operators apply coatings using an electrocoat (“e-coat” or “e-coating”) process. The process of applying an e-coat can involve several steps. In a first step (“pretreatment”), an operator will clean the substrate (e.g., a metal substrate), and apply a material such as phosphate, to prepare the surface for application of the e-coat. In a next step, an operator can apply coatings to the pretreated substrate in an electrocoat bath using calibrated process control equipment. The e-coat bath may consist of certain compositional metrics, such as a certain percentage of solvent, with a remaining percentage of paint solids, where the solvent may serve as a carrier for the paint solids. Key compositional metrics of the e-coat bath, including but not limited to the percentage of solids, as well as the pigment to binder ratio, may be important in some cases to maintain within an acceptable range.

Maintaining an ideal range of such compositional metrics can be fairly time and effort intensive. In one example, maintaining an ideal range of, for example, percentage solids, or pigment-to-binder ratio, etc., may involve an operator continually obtaining samples of the e-coat bath, and analyzing the samples. The analysis, in turn, may take several hours or days to complete, which can hold up production.

### BRIEF SUMMARY

The present disclosure provides systems, methods, and computer program products for efficiently and accurately maintaining an appropriate level of compositional metrics, including but not limited to percentage of solids, and/or pigment-to-binder ratio in an e-coat bath. For example, a system may contain multiple sensors that may be applied within (or connected directly to/in-line with) an e-coat bath. The one or more sensors are selected to identify traits of the e-coat bath that correlate well with the compositional metrics. An exemplary system can apply data from the various inputs from the e-coat bath, such as by using one or more algorithms, including one or more machine learning algorithms, to identify if certain of the compositional metrics have changed. An operator, or the system itself, can then make appropriate real-time adjustments to the solution to ensure corresponding real-time optimization of the compositional metrics in the e-coat bath.

For example, a computer-implemented method (and corresponding computer system configured to implement the same) for automatically maintaining compositional metric values of the e-coat bath, can include receiving initial data for an e-coat bath from a sensor, wherein initial data corresponds to a compositional metric value of the e-coat bath, the composition metric comprising one or both of (i) percent solids, and (ii) pigment-to-binder ratio. The method can also include processing the initial data using an algorithm to identify an initial compositional metric value of the e-coat bath. In addition, the method can include determining, by the computer system from the initial compositional metric value, one or more amounts of material to be added to the e-coat bath. Furthermore, the method can include receiving subsequent data from the sensor, and processing the received subsequent data with the one or more algorithms to identify a subsequent compositional metric value of the e-coat bath.

Additional features and advantages will be set forth in the description which follows, and in part will be obvious from the description, or may be learned by practice. The features and advantages may be realized and obtained by means of the instruments and combinations particularly pointed out in the appended claims and aspects. These and other features will become more fully apparent from the following description and appended claims, or may be learned by the practice of the examples as set forth hereinafter.

### BRIEF DESCRIPTION OF THE DRAWINGS

In order to describe the manner in which the above recited and other advantages and features can be obtained, a more particular description briefly described above will be rendered by reference to specific examples thereof, which are illustrated in the appended drawings. Understanding that these drawings are merely illustrative and are not therefore to be considered to be limiting of its scope, the present disclosure will be described and explained with additional specificity and detail through the use of the accompanying drawings in which:

FIG. 1 illustrates a schematic diagram in which a server computer interacts with sensor data to maintain key compositional metrics of an e-coat bath;

FIG. 2 illustrates a graph of one composition metric, namely percent solids, in an e-coat bath in view of monitoring and adjusting for sensor data;

FIG. 3 illustrates a graph of another composition metric, namely pigment-to-binder ratio. in an e-coat bath in view of monitoring and adjusting for sensor data; and

FIG. 4 illustrates a flowchart of a series of acts in a method of automatically maintaining percent solids in an e-coat bath.

### DETAILED DESCRIPTION

The present disclosure provides systems, methods, and computer program products for efficiently and accurately maintaining an appropriate level of compositional metrics, including but not limited to percentage of solids, and/or pigment-to-binder ratio in an e-coat bath. For example, a system may contain multiple sensors that may be applied within (or connected directly to/in-line with) an e-coat bath. The one or more sensors are selected to identify traits of the e-coat bath that correlate well with the compositional metrics. An exemplary system can apply data from the various inputs from the e-coat bath, such as by using one or more algorithms, including one or more machine learning algorithms, to identify if certain of the compositional metrics have changed. An operator, or the system itself, can then make appropriate real-time adjustments to the solution to ensure corresponding real-time optimization of the compositional metrics in the e-coat bath.

Accordingly, as will be understood more fully herein, the present disclosure can provide significant time and cost savings for e-coat processes. For example, e-coat bath monitoring processes that normally would halt production for hours or days to monitor and determine the percent solids in an e-coat bath can now be done far more quickly than previously, and can be done in real-time without stopping production. As such, the disclosed systems can accomplish the enhanced speed without sacrificing accuracy, and using a wide range of data available from readily available sensors.

As a preliminary matter, unless otherwise expressly specified, all numbers, such as those expressing values, ranges, amounts, or percentages, may be read as if prefaced by the word “about,” even if the term does not expressly appear. Any numerical range recited herein is intended to include all sub-ranges subsumed therein. The plural encompasses the singular and vice versa. For example, while the present disclosure has been described in terms of “a” component, “an” algorithm, “a” layer, “a” sensor, “a” pigment, or the like, more than one of these and other components, including mixtures, can be used. When ranges are given, any endpoints of those ranges and/or numbers within those ranges can be combined with the scope of the present disclosure. Likewise, the terms “including,” “such as,” “for example,” or the like are to be understood as open-ended, meaning “including/such as/for example but not limited to.”

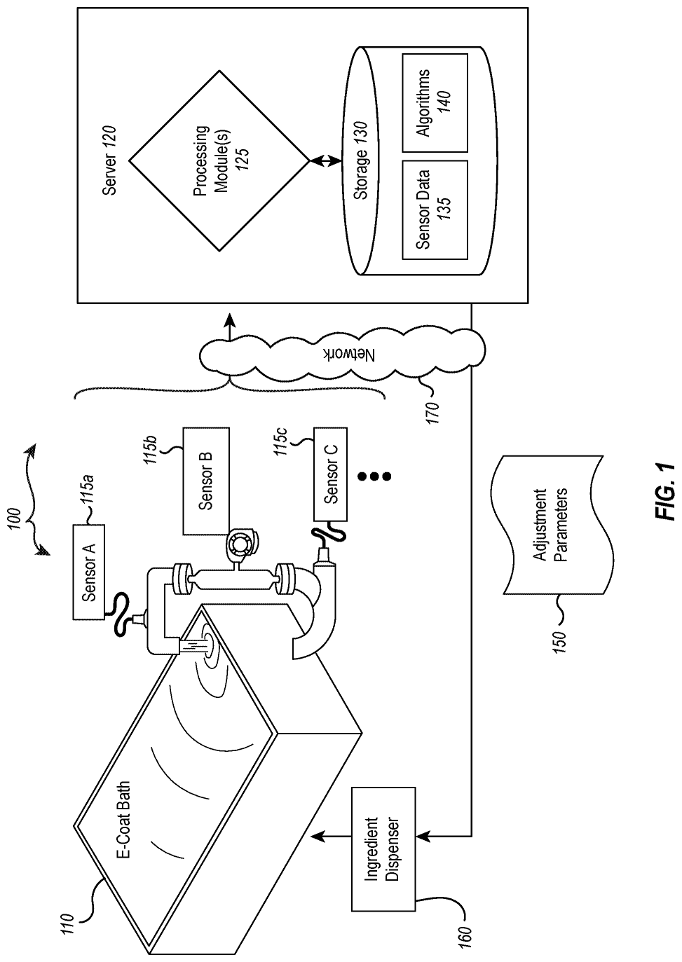

Referring now to the Figures, FIG. 1 illustrates a generalized schematic in which a server computer 120 interacts with sensor data to maintain a compositional metric of an e-coat bath. In particular, FIG. 1 shows that a system 100 can comprise an e-coat bath 110, to which is connected a series of sensors 115, namely the illustrated Sensor A 115a, Sensor B 115b, Sensor C 115c, and so on. The e-coat bath 110 can comprise any number of compositional ingredients used to coat a substrate, such a paint, basecoat, or other protective coating/layer. In one example, the e-coat bath comprises about 80-90% deionized water and 10-20% paint solids. The substrate, in turn, can comprise for example a metal substrate for use in an automotive fabrication environment.

As noted above, FIG. 1 further shows that e-coat bath 110 can comprise any number of sensors A, B, C (i.e., 115a, 115b, 115c) connected directly thereto. By way of explanation, although FIG. 1 illustrates three sensors, the present disclosure contemplates use of at least one of the illustrated sensors, and may further implement more than three sensors connected to the e-coat bath 110. In one example, the sensors 115(a-c, etc.) can include any one or more sensors configured to measure any one or more of the following parameters, or “compositional metrics”: pigment-to-binder (or “P/B”), pH, conductivity, density, viscosity, turbidity, temperature, bath level, bath volume, product flow rate, biocide levels, sulfuric acid, lactic acid, and/or bacterial growth.

By way of example and not of limitation, an exemplary pH sensor can comprise a sensor model CPS61E made by ENDRESS+HAUSER. In this or another example, the pH sensor (e.g., 115a) can comprise one or more ceramic reference junction pH probes, mounted in-line with range from 0-14 pH. In one example, the pH sensor can be mounted in-line with an ENDRESS+HAUSER fitting (e.g., CPA871) to allow for removal of the probe for calibration/cleaning while e-coating process continues to run. By way of example and not of limitation, a conductivity sensor can comprises a sensor model CLS82E made by ENDRESS+HAUSER. In one example, the conductivity sensor (e.g., 115b) comprises a 4-point conductivity probe that is mounted in-line with the above-noted ENDRESS+HAUSER CPA871 fitting to allow for removal of probe for calibration/cleaning while process continues to run.

By way of example and not of limitation, a density and viscosity sensor can comprise an in-line Coriolis flow meter, such as made by ENDRESS+HAUSER. In one example, the density and viscosity sensors (e.g., 115c) can comprise models PROMASS Q 300, and/or PROMASS I 300. By way of further example and not of limitation, at least one implementation of a turbidity sensor (e.g., 115d) is model CUS52D made by ENDRESS+HAUSER. As discussed with the prior sensors, the turbidity sensor can be mounted in-line, thereby allowing for removal while processes continue to run. In addition to the foregoing, and although not illustrated, the server 120 can include or otherwise be operably connected with one or more programmable logic controllers (PLC), such as a model ILC2050BI PLC that provides a variable sample rate. In at least one example, the one or more PLC can interface between the one or more sensors 115(a-c, etc.) and the server 120. In one example, pigment-to-binder ratio can be derived from turbidity data. In another example, pigment-to-binder ratio can be reasonably well estimated without turbidity, in this case using only density, viscosity, and conductivity. One will appreciate in view of the specification that both percent solids and pigment-to-binder ratio can be derived from one or multiple of the sensors listed herein, thus providing flexibility with the ability to mix-and-match sensors on an as-needed basis.

FIG. 1 further shows that server 120 can send and receive data from each of the given sensors 115(a-c) and/or the ingredient dispenser 160, discussed more fully below, such as via a direct connection, and/or via a network 170. The data that is sent and received, in turn, can be handled by the server 120 through the one or more processing modules 125. The one or more processing modules 125 can interact with storage 130, and pass or retrieve information from sensor data component 135, and algorithm component 140.

By way of explanation, the term “module” as used herein means computer executable code that, when executed by one or more processors at a given computer system (e.g., sever 120), causes the given computer system to perform a particular function. By contrast, the term “component,” as used herein, means a passive set of instructions or data structures that store, manage, and/or otherwise provide information handled or otherwise processed through a given module. One of skill in the art, however, will appreciate that the distinction between different modules or components is at least in part arbitrary, and that modules or components may be otherwise combined and/or divided and still remain within the scope of the present disclosure. As such, the description of a given element as being a “module” or a “component” is provided only for the sake of clarity and explanation and should not be interpreted to indicate that any particular structure of computer executable code and/or computer hardware is required, unless expressly stated otherwise. In this description, the terms “component,” “agent,” “manager,” “service,” “engine,” “virtual machine” or the like may also similarly be used.

Referring again to FIG. 1, the one or more processing modules 125 of server 120 can identify the level of percent solids in the e-coat bath 110 by calculating the data received from the various sensors (115a, 115b, and 115c). In one example, the one or more processing modules 125 load one or more algorithms from component 140. The one or more algorithms can include one or more machine learning algorithms. The one or more machine learning algorithms can further employ one or more mathematical formulations to identify correlations between sensor data and key compositional metrics, such as percent solids, or pigment-to-binder ratio.

With respect to percent solids, for example, the computer system 120 may employ statistical modeling algorithms to identify whether the e-coat bath is in line with expectations, or out of line. An exemplary statistical modeling algorithm that is suitable for use with the present disclosure includes use of a multiple linear regression algorithm represented by the following equation: Y=β₀+β₁X₁+ . . . βₙXₙ, where β₀ is a graph intercept value, each of β₁ through βₙ comprises a coefficient value, and the values of X₁ through Xₙ represent raw values taken from the respective sensor. Y=β₀+Σₙ₌₁^{N}βₙXₙ

In general, the coefficient values of β₁ through βₙ essentially normalize the scale of the sensor 115(a-c) values so that the values can be added together in a meaningful way to achieve a meaningful Y result value (i.e., reflecting the percent solids in the e-coat bath 110 solution). The following Table I provides an example set of coefficients (and intercept) that may be used in accordance with the present disclosure.

TABLE I

| Sensor Type | β - coefficients |
|---|---|
| Density (g/mL) | 0.4358668 |
| Viscosity (cP, @ 25° C.) | 0.03346202 |
| pH | 0.1349582 |
| Conductivity (μS/cm) | 2.72E−06 |
| Intercept | −1.145718 |

Thus, by way of example, assuming β₁X₁ represents the subset for pH, and substituting for a pH of 7.0, the equation may take the form of:

$$Y = - 1 \text{.145718} + ( 0.13 \; 49582 ) ( 7.0 ) + … {β}_{n} {X}_{n}$$

By way of explanation, the values of FIG. 1 are examples of processed rather than raw sensor data, but nevertheless may be used directly in the model. For example, raw data taken from sensors 115(a-c) may in some cases be temperature-corrected/adjusted to counteract minor process temperature variation before being applied within a given algorithm. Thus, in some cases, raw sensor 115(a-c) data may be used, while in other cases data may be used that has been adjusted for local conditions.

FIG. 1 further shows that server 120 can, upon identification of the results of the calculations, send one or more messages 150 to an ingredient dispenser 160 to an adjust an amount of material. In general, the threshold for what constitutes in or out of range will be recipe dependent. With respect to percent solids, some operators with certain coatings and substrates will desire a percent solids concentration of between 1% and 4% for various reasons, while others may desire an ideal compositional metrics, such as percent solids concentration, will be between 15%-18% and 24%-50%. Similarly, the specification range for pigment to binder ratio may vary based on process, coating product, and desired coating properties. Thus, one will understand that the desired threshold range (or “threshold”) provided to the server 120 can be dependent on a number of different considerations, including but not limited to customer preferences, end application, substrate to be coated, desired e-coat recipe ingredients, and can further include a variety of other considerations.

Accordingly, in one example, message 150 can include a generalized value of the percent solids (or other material adjustments) that ingredient dispenser 160 (e.g., a dosing pump) can interpret for determining the right amount of material to add or adjust in the solution. In such a case, ingredient dispenser 160 can receive and interpret message 150, and compare the indication of adjustment with the customer-requested values for the compositional metrics (percent solids, P/B ratio) of the current solution in e-coat bath 110. The ingredient dispenser 160 may then provide a relevant amount of material to adjust the solution.

For example, the dispenser 160 may deliver an effective amount “material” defined as any one or more of the following: e-coat paste, e-coat resin, and formic acid via a dosing pump. In another example, server 120 performs those same calculations and threshold evaluation steps via processors 125, so that message 150 includes specific instructions about what material to add, how much, and so on. In that case, ingredient dispenser 160 merely executes the instructions from message 150 and adjusts the e-coat bath 110. In at least one example, the server 120, e-coat bath 110, and ingredient dispenser 160 are in continual communication, and thus continually monitoring and adjusting the e-coat bath 110 to optimize percent solids within the acceptable range in real-time.

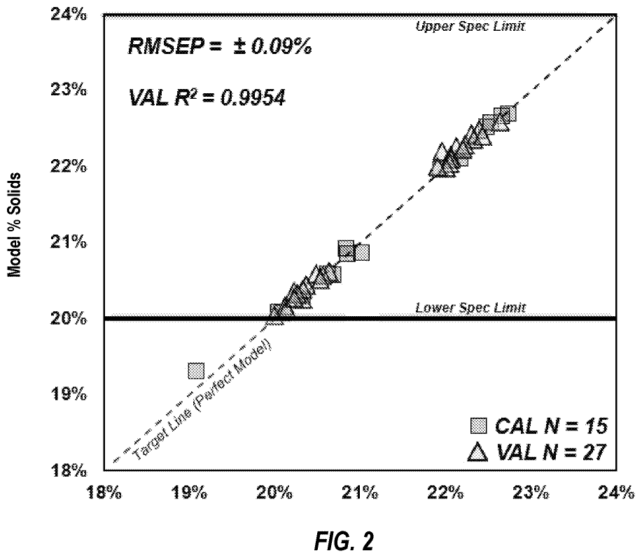

FIG. 2 illustrates a graph indicating percent solids as an exemplary proof of concept, where the Y axis considers calculated values measured from the c-coat bath 110, while the X axis shows manually measured values. As shown, the modeled values (Y axis) are essentially identical to the actual, measured values (X values) for the same points in time.

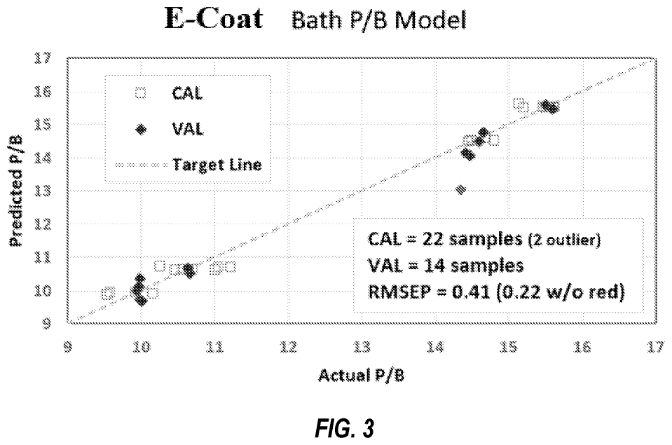

By contrast, FIG. 3 illustrates a graph showing pigment-to-binder (P/B) ratio in an e-coat bath. Similar to FIG. 2, the plot shows a similar relationship in terms of predicting P/B ratio using the same or similar multivariate approach used above for % solids in FIG. 2. In other words, multiple linear regression modeling of P/B can be derived from sensors 115(a-c) that sense conductivity, density, and viscosity. The server 120 can estimate P/B+/−0.41 vs. spec range of 10-15 across all validation samples. The case illustrated in FIG. 3 shows that there were two outliers on the calibration set, and one outlier on the validation set, all of which seemed to fall near the start of the experiment. Once the experiment was in full swing and the process was stable, FIG. 3 shows that the root mean square error conformed with +/−0.22 P/B vs. spec range of 10-15.

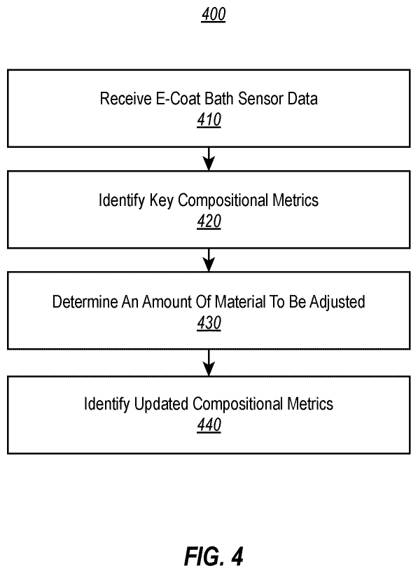

Accordingly, FIGS. 1-3 provide numerous components, modules, and schematics that can be used for accurately monitoring and adjusting key compositional metrics of an e-coat bath in real-time. The present disclosure can also be described in terms of acts in a method of accomplishing a particular result. The method of FIG. 4 is described below with respect to elements, components, and modules of FIGS. 1-3.

For example, FIG. 4 shows that a method 400 of automatically maintaining percent solids in an e-coat bath can comprise an act 410 of receiving (or identifying) e-coat bath sensor data, or other relevant properties. Act 410 includes receiving initial data for an e-coat bath from a sensor, wherein initial data corresponds to a compositional metric of the e-coat bath, the composition metric comprising one or both of (i) percent solids, and (ii) pigment-to-binder ratio. For example, FIG. 1 shows that server 120 measures the properties of e-coat bath 110 via data from one or more of sensors 115a, 115b, 115c, and/other sensors or combinations thereof, as discussed herein.

FIG. 4 also shows that method 400 can comprise act 420 of identifying key compositional metrics for the e-coat bath. Act 420 includes processing the initial data using an algorithm to identify an initial compositional metric value of the e-coat bath. For example, FIG. 1 shows that server 120 processes the sensor 115 data in connection with one or more algorithms 140. The one or more algorithms 140 can employ any number or type of statistical modeling algorithms to process sensor data that is determined to correlate with percent solids. The present disclosure contemplates that one or more or all of the following sensor data points can be analyzed individually or in various combinations as correlating with key compositional metrics: (i) Pigment to Binder; (ii) pH (iii) conductivity; (iv) density; (v) viscosity; (vi) turbidity; (vii) temperature; (viii) bath level; (ix) bath volume; (x) product flow rate; (xi) biocide levels; (xii) sulfuric acid; (xiii) lactic acid, (xiv) bacterial growth. In at least one example, temperature may be combined with viscosity measurements. In yet another example, the sensors employed include pH, conductivity, density, and viscosity (which may be combined with temperature).

In addition, FIG. 4 shows that method 400 can comprise an act 430 of determining an amount of material to be adjusted/added. Act 430 includes determining, by the computer system from the initial compositional metric value, one or more amounts of material to be added to the e-coat bath. For example, FIG. 1 shows that server 120 sends one or more messages 150 to an ingredient dispenser 160. The one or more messages 150 may comprise the general information regarding calculated percent solids, which enables the ingredient dispenser 160 to separately determine an e-coat bath recipe-specific solution of material to be added. Alternatively, message 150 can include specific instructions to dispense a specific material into the e-coat bath via ingredient dispenser 160. In still another case, the message 150 is displayed on a client device, such as a local or attached personal computer attached to the e-coat bath 110, or any number of other mobile devices including a laptop, mobile phone, or tablet, etc. The operator can then take the displayed information to manually dispense an appropriate amount of material.

Furthermore, FIG. 4 shows that method 400 can comprise an act 440 of identifying an updated compositional metrics. Act 440 includes receiving subsequent data from the sensor, and processing the received subsequent data with the one or more algorithms to identify a subsequent compositional metric value of the e-coat bath. For example, as shown in FIG. 1, since the connections and measurements between bath 110, sensors 115, server 120, and dispenser 160 may be continuous and ongoing, server 120 can continually calculate in real-time the relevant material to adjust (e.g., to adjust the percent solids or pigment-to-binder ratio), and make appropriate ongoing adjustments, all without halting ongoing e-coating processes.

Accordingly, FIGS. 1-4 provide a number of systems, components, and modules that can be used to significantly increase both speed and accuracy of e-coating bath maintenance of percent solids. Part of this increase can be provided by use of monitoring a wide number of data points that correlate closely with changes in percent solids in solution. Thus, the principles outlined in this disclosure can provide end users with significantly more efficient and accurate tools to maintain e-coat production lines without sacrificing quality.

The present disclosure may comprise or utilize a special-purpose or general-purpose computer system that includes computer hardware, such as, for example, one or more processors and system memory, as discussed in greater detail below. The scope of the present disclosure also includes physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the disclosure may be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

A cloud-computing environment, or cloud-computing platform, may comprise a system that includes one or more hosts that are each capable of running one or more virtual machines. During operation, virtual machines emulate an operational computing system, supporting an operating system and perhaps one or more other applications as well. Each host may include a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth. In view of the foregoing, the present disclosure may be embodied in multiple different configurations, as outlined above, and as exemplified by the following configurations or aspects.

For example, at least one exemplary configuration can include a computer-implemented method for use in a system that includes a computer system, an e-coat bath, a network, and one or more sensors connected thereto, the method for automatically maintaining compositional metrics of the e-coat bath, with the method comprising: receiving initial data for an e-coat bath from a sensor, wherein initial data corresponds to a compositional metric value of the e-coat bath, the composition metric comprising one or both of (i) percent solids, and (ii) pigment-to-binder ratio; processing the initial data using an algorithm to identify an initial compositional metric value of the e-coat bath; determining, by the computer system from the initial compositional metric value value, one or more amounts of material to be added to the c-coat bath; and receiving subsequent data from the sensor, and processing the received subsequent data with the one or more algorithms to identify a subsequent compositional metric value of the e-coat bath.

In an additional or alternative configuration, the one or more sensors comprise a density sensor connected to the e-coat bath. In an additional or alternative configuration, the one or more sensors comprise a viscosity sensor connected to the e-coat bath. In an additional or alternative configuration, the one or more sensors include a pH sensor connected to the e-coat bath. In an additional or alternative configuration, the one or more sensors include a conductivity sensor connected to the e-coat bath. In an additional or alternative configuration: the sensor includes a turbidity sensor connected to the e-coat bath; and the computer system using the algorithm to calculate pigment-to-binder ratio from the data obtained from the turbidity sensor. In an additional or alternative configuration, the one or more sensors include a temperature sensor connected to the e-coat bath. In an additional or alternative configuration, the one or more sensors include a bath level sensor connected to the e-coat bath. In an additional or alternative configuration, the one or more sensors include a bath volume sensor connected to the e-coat bath.

Furthermore, in an additional or alternative configuration, the one or more sensors include a product flow rate sensor connected to the e-coat bath. In an additional or alternative configuration, the initial compositional metric value indicates that a percent solids is outside of a threshold range. In an additional or alternative configuration, the initial compositional metric value indicates that a pigment-to-binder ratio is outside of a desired threshold range. In an additional or alternative configuration, the method can further comprise one or more of: displaying a determined amount of material to be added to the e-coat bath on a digital display; and/or sending, via the computer system, an instruction to an ingredient dispenser to dispense the determined one or more amounts of material to the e-coat bath. In an additional or alternative configuration: the material dispensed by the ingredient dispenser comprises a make-up material comprising any one or more of an e-coat paste, an e-coat resin, and/or formic acid; and the ingredient dispenser comprises a dosing pump.

Still further, in an additional or alternative configuration, the method can further comprise: using a machine learning algorithms to perform the steps of: processing the received sensor data, and determining the amount of material to be added to the e-coat bath. In an additional or alternative configuration: the step of processing comprises use of a multiple linear regression algorithm having the following equation: Y=β_0+β_1 X_1+ . . . β_n X_n and each of X_1 through X_n comprises a value taken from the sensor.

In addition to the foregoing, another configuration of the present disclosure can include a computer system comprising: one or more processors; one or more computer-readable storage media having stored thereon computer-executable instructions that, when executed by the one or more processors, cause the computer system to perform the following: receive first sensor data from a sensor, wherein the sensor is configured to identify properties about an e-coat bath that correlate with a compositional metric value in the e-coat bath, wherein the compositional metric value includes a measure of percent solids, or pigment-to-binder ratio; process the received sensor data by calculating the received sensor data in line with one or more algorithms to identify an initial compositional metric value in the e-coat bath; determine from the initial compositional metric value an amount of material to be adjusted within the e-coat bath; and receive second sensor data from one or more sensors, and process the received second sensor data with the algorithm to identify a subsequent compositional metric value corresponding to an updated material in the e-coat bath. In an additional or alternative configuration, the sensor comprises any one or more of a the following connected to the e-coat bath: a density sensor, a viscosity sensor, a pH sensor, a conductivity sensor, a turbidity sensor, a temperature sensor, a bath level sensor, a bath volume sensor, and a product flow rate sensor.

In an additional or alternative configuration: the sensor is mounted in-line with a fitting; and the fitting is configured to allow removal of the sensor for calibration and/or cleaning while the e-coat bath continues to run. In an additional or alternative configuration, the initial percent solids is outside of a desired threshold range. In an additional or alternative configuration, the updated percent solids is within a desired threshold range. In an additional or alternative configuration, the computer-executable instructions, when executed by the one or more processors, further cause the computer system to perform one or more of following: display the determined one or more amounts of material to be adjusted to the e-coat bath on a digital display; and/or send one or more instructions to an ingredient dispenser that indicate the determined material to be added to the e-coat bath. In an additional or alternative configuration, the method can further comprise: one or more machine learning algorithms that, when executed by the computer system, perform the steps of processing the received sensor data, and determining an amount of material needed to adjust the e-coat bath. In an additional or alternative configuration: the step of processing the received sensor data by calculating the received sensor data further comprises use of a multiple linear regression algorithm having the following equation: Y=β_0+β_1 X_1+ . . . β_n X_n and each of X_1 through X_n comprises a value taken from one or the one or more sensors.

The present disclosure may also be enumerated as exemplary aspects, which may be combined or substituted with the above-listed configurations in any variation or format. For example, in one aspect, a computer-implemented method for automatically maintaining percent solids in an e-coat bath can include: receiving first sensor data from one or more sensors, wherein the one or more sensors are configured to identify properties about an e-coat bath that correlate with percent solids in the e-coat bath; processing the received sensor data by calculating the received sensor data in line with one or more algorithms to identify an initial value corresponding to an initial percent solids in the e-coat bath; determining from the initial value one or more amounts of material that need to be added to the e-coat bath; and receiving second sensor data from one or more sensors, and processing the received second sensor data with the one or more algorithms to identify a subsequent value corresponding to an updated percent solids in the e-coat bath. In a second aspect, in the computer-implemented method as recited in the first aspect, the one or more sensors comprise a density sensor connected to an e-coat bath.

In a third aspect, in the computer-implemented method as recited in any of the preceding aspects, the one or more sensors comprise a viscosity sensor connected to an e-coat bath. In a fourth aspect, in the computer-implemented method as recited in any of the preceding aspects, the one or more sensors include a pH sensor connected to an e-coat bath. In a fifth aspect, in the computer-implemented method as recited in any of the preceding aspects, the one or more sensors include a conductivity sensor connected to an e-coat bath. In a sixth aspect, in the computer-implemented method as recited in any of the preceding aspects, the one or more sensors include a turbidity sensor connected to an e-coat bath. In a seventh aspect, in the computer-implemented method as recited in any of the preceding aspects, the one or more sensors include a temperature sensor connected to an e-coat bath. In an eight aspect, in the computer-implemented method as recited in any of the preceding aspects, the one or more sensors include a bath level sensor connected to an e-coat bath.

In a ninth aspect, in the computer-implemented method as recited in any of the preceding aspects, the one or more sensors include a bath volume sensor connected to an e-coat bath. In a tenth aspect, in the computer-implemented method as recited in any of the preceding aspects, the one or more sensors include a product flow rate sensor connected to an e-coat bath. In an eleventh aspect, in the computer-implemented method as recited in any of the preceding aspects, the initial compositional metrics, such as percent solids (and/or pigment-to-binder ratio) is outside of a threshold range. In a twelfth aspect, in the computer-implemented method as recited in any of the preceding aspects, the updated compositional metrics, such as percent solids (and/or pigment-to-binder ratio) is within the desired threshold range. In a thirteenth aspect, in the computer-implemented method as recited in any of the preceding aspects, a server computer receives and processes the initial and subsequent sensor data. In a fourteenth aspect, the computer-implemented method as recited in any of the preceding aspects can further include one or more of: displaying the determined one or more amounts of material that need to be added to the e-coat bath on a digital display; and/or sending one or more messages that indicate the determined one or more amounts of material that need to be added to the e-coat bath to an ingredient dispenser.

In a fifteenth aspect, in the computer-implemented method as recited in any of the preceding aspects: the solid dispensed by the ingredient dispenser comprises a make-up material comprising any one or more of an e-coat paste, an e-coat resin, and/or formic acid; and the ingredient dispenser comprises a dosing pump. In a sixteenth aspect, the computer-implemented method as recited in any of the preceding aspects can further include using one or more machine learning algorithms to perform the steps of: processing received sensor data, and determining an amount of materials to be added to the e-coat bath. In a seventeenth aspect, in the computer-implemented method as recited in any of the preceding aspects: the step of processing the received sensor data by calculating the received sensor data further comprises use of a multiple linear regression algorithm having the following equation: Y=β₀+β₁X₁+ . . . βₙXₙ and each of X₁ through Xₙ comprises a value taken from one or the one or more sensors. By way of further example, β₁ through βₙ comprise corresponding coefficients, including normalization coefficients, to the values of X₁ through Xₙ.

In an eighteenth aspect, in an additional or alternative configuration of the present disclosure, a computer system can include one or more processors; one or more computer-readable storage media having stored thereon computer-executable instructions that, when executed by the one or more processors, cause the computer system to perform the following: receive first sensor data from one or more sensors, wherein the one or more sensors are configured to identify properties about an e-coat bath that correlate with percent solids in the e-coat bath; process the received sensor data by calculating the received sensor data in line with one or more algorithms to identify an initial value corresponding to an initial percent solids in the e-coat bath; determine from the initial value one or more amounts of material that need to be added to the e-coat bath; and receive second sensor data from one or more sensors, and process the received second sensor data with the one or more algorithms to identify a subsequent value corresponding to an updated percent solids in the e-coat bath.

In a nineteenth aspect, in the computer system as recited in the nineteenth aspect, the one or more sensors comprise a density sensor connected to an e-coat bath. In a twentieth aspect, in the computer system as recited in any of the preceding aspects eighteen through nineteen, the one or more sensors comprise a viscosity sensor connected to an e-coat bath. In a twenty-first aspect, in the computer system as recited in any of the preceding aspects eighteen through twenty, the one or more sensors include a pH sensor connected to an e-coat bath. In a twenty-second aspect, in the computer system as recited in any of the preceding aspects eighteen through twenty one, the one or more sensors include a conductivity sensor connected to an e-coat bath. In a twenty-third aspect, in the computer system as recited in any of the preceding aspects eighteen through twenty-two, the one or more sensors include a turbidity sensor connected to an e-coat bath.

In a twenty-fourth aspect, in the computer system as recited any of the eighteenth through twenty-third aspects, the one or more sensors include a temperature sensor connected to an e-coat bath. In a twenty-fifth aspect, in the computer system as recited in of the preceding eighteenth through twenty-fourth aspects, the one or more sensors include a bath level sensor connected to an e-coat bath. In a twenty-sixth aspect, in the computer system as recited in the preceding eighteenth through twenty-seventh aspects, the one or more sensors include a bath volume sensor connected to an e-coat bath. In a twenty-seventh aspect, in the computer system as recited in any of the preceding eighteenth through twenty-sixth aspects, the one or more sensors include a product flow rate sensor connected to an e-coat bath.

In a twenty-eighth aspect, in the computer system as recited in any of the preceding eighteenth through twenty-seventh aspects, the one or more sensors are mounted in-line with a fitting figured to allow removal of the one or more sensors for calibration and/or cleaning while the e-coat bath continues to run. In a twenty-ninth aspect, in the computer system as recited in the preceding eighteenth through twenty-eighth aspects, the initial percent solids is outside of a desired threshold range. In a thirtieth aspect, in the computer system as recited in any of the preceding eighteenth through twenty-ninth aspects, the updated percent solids is within a desired threshold range.

In a thirty-first aspect, in the computer system as recited in any of the preceding eighteenth through thirtieth aspects, the computer-executable instructions, when executed by the one or more processors, further cause the computer system to perform one or more of following: display the determined one or more amounts of material that need to be added to the e-coat bath on a digital display; and/or send one or more messages that indicate the determined one or more amounts of material that need to be added to the e-coat bath to an ingredient dispenser. In a thirty-second aspect, in the computer system as recited in any of the preceding eighteenth through thirty-first aspects, the computer system can further include: one or more machine learning algorithms that, when executed by the computer system, perform the steps of processing received sensor data, and determining an amount of materials to be added to the e-coat bath. In a thirty-third aspect, in the computer system as recited in any of the preceding eighteenth through thirty-second aspects: the step of processing the received sensor data by calculating the received sensor data further comprises use of a multiple linear regression algorithm having the following equation: Y=β₀+β₁X₁+ . . . βₙXₙ and each of X₁ through Xₙ comprises a value taken from one or the one or more sensors. By way of further example, β₁ through βₙ comprise corresponding coefficients, including normalization coefficients, to the values of X₁ through Xₙ.

In a thirty-second aspect, in the computer-implemented method as recited in any of the aspects one to seventeen: the determination of one or more amounts of material to be added to the e-coat bath considers that the (i) percent solids, and/or (ii) pigment-to-binder ratio of the e-coat is to be brought to the desired threshold range, such as in the desired threshold range of the desired e-coat recipe.

In a thirty-third aspect, in the computer-implemented method as recited in any of the aspects one to seventeen or thirty-three: the method for automatically maintaining compositional metrics of the e-coat bath is carried out continuously during a process of coating multiple substrates.

Although the subject matter has been described in language specific to structural features and/or methodological acts, and whereas particular examples of this disclosure have been described above for purposes of illustration, it will be evident to those skilled in the art that numerous variations of the details of the present disclosure may be made without departing from what is defined in the appended claims. In particular, it is to be understood that the subject matter defined in the appended claims is not necessarily limited to the described features or acts described above, or the order of the acts described above. Rather, the described features and acts are disclosed as example forms of implementing the claims.

We claim:

1. A computer-implemented method for use in a system that includes a computer system, an e-coat bath, a network, and one or more sensors connected thereto, the method for automatically maintaining compositional metric values of the e-coat bath, comprising:
receiving initial data for an e-coat bath from a sensor, wherein initial data corresponds to a compositional metric value of the e-coat bath, the composition metric comprising one or both of (i) percent solids, and (ii) pigment-to-binder ratio;
processing the initial data using an algorithm to identify an initial compositional metric value of the e-coat bath;
determining, by the computer system from the initial compositional metric value, one or more amounts of material to be added to the e-coat bath; and
receiving subsequent data from the sensor, and processing the received subsequent data with the one or more algorithms to identify a subsequent compositional metric value of the e-coat bath.

2. The computer-implemented method as recited in claim 1, wherein the one or more sensors comprise a density sensor connected to the e-coat bath.

3. The computer-implemented method as recited in claim 1, wherein the one or more sensors comprise a viscosity sensor connected to the e-coat bath.

4. The computer-implemented method as recited in claim 1, wherein the one or more sensors include a pH sensor connected to the e-coat bath.

5. The computer-implemented method as recited in claim 1, wherein the one or more sensors include a conductivity sensor connected to the e-coat bath.

6. The computer-implemented method as recited in claim 1, wherein:
the sensor includes a turbidity sensor connected to the e-coat bath; and
the computer system using the algorithm to calculate pigment-to-binder ratio from the data obtained from the turbidity sensor.

7. The computer-implemented method as recited in claim 1, wherein the one or more sensors include a temperature sensor connected to the e-coat bath.

8. The computer-implemented method as recited in claim 1, wherein the one or more sensors include a bath level sensor connected to the e-coat bath.

9. The computer-implemented method as recited in claim 1, wherein the one or more sensors include a bath volume sensor connected to the e-coat bath.

10. The computer-implemented method as recited in claim 1, wherein the one or more sensors include a product flow rate sensor connected to the e-coat bath.

11. The computer-implemented method as recited in claim 1, wherein the initial compositional metric value indicates that a percent solids is outside of a threshold range.

12. The computer-implemented method as recited in claim 1, wherein the initial compositional metric value indicates that a pigment-to-binder ratio is outside of a desired threshold range.

13. The computer-implemented method as recited in claim 1, further comprising one or more of:
displaying a determined amount of material to be added to the e-coat bath on a digital display; and/or
sending, via the computer system, an instruction to an ingredient dispenser to dispense the determined one or more amounts of material to the e-coat bath.

14. The computer-implemented method as recited in claim 13, wherein:
the material dispensed by the ingredient dispenser comprises a make-up material comprising any one or more of an e-coat paste, an e-coat resin, and/or formic acid; and
the ingredient dispenser comprises a dosing pump.

15. The computer-implemented method as recited in claim 1, further comprising:
using a machine learning algorithms to perform the steps of:
processing the received sensor data, and
determining the amount of material to be added to the e-coat bath.

16. The computer-implemented method as recited in claim 1, wherein:
the step of processing comprises use of a multiple linear regression algorithm having the following equation:
$$Y = {β}_{0} + {β}_{1} {X}_{1} + … {β}_{n} {X}_{n}$$
and
each of X₁ through Xₙ comprises a value taken from the sensor.

17. A computer system comprising:
one or more processors;
one or more computer-readable storage media having stored thereon computer-executable instructions that, when executed by the one or more processors, cause the computer system to perform the following:
receive initial data for an e-coat bath from a sensor, wherein initial data corresponds to a compositional metric value of the e-coat bath, the composition metric comprising one or both of (i) percent solids, and (ii) pigment-to-binder ratio;
process the initial data using an algorithm to identify an initial compositional metric value of the e-coat bath;
determine, by the computer system from the initial compositional metric value, one or more amounts of material to be added to the e-coat bath; and
receive subsequent data from the sensor, and processing the received subsequent data with the one or more algorithms to identify a subsequent compositional metric value of the e-coat bath;
wherein the sensor comprises any one or more of a the following connected to the e-coat bath: a density sensor, a viscosity sensor, a pH sensor, a conductivity sensor, a turbidity sensor, a temperature sensor, a bath level sensor, a bath volume sensor, and a product flow rate sensor.

18. (canceled)

19. The computer system as recited in claim 17, wherein:
the sensor is mounted in-line with a fitting; and
the fitting is configured to allow removal of the sensor for calibration and/or cleaning while the e-coat bath continues to run.

20. The computer system as recited in claim 17, wherein the initial percent solids is outside of a desired threshold range.

21. (canceled)

22. The computer system as recited in claim 17, wherein the computer-executable instructions, when executed by the one or more processors, further cause the computer system to perform one or more of following:
display the determined one or more amounts of material to be adjusted to the e-coat bath on a digital display; and/or
send one or more instructions to an ingredient dispenser that indicate the determined material to be added to the e-coat bath;
one or more machine learning algorithms that, when executed by the computer system, perform the steps of
processing the received sensor data,
determining an amount of material needed to adjust the e-coat bath; and
the step of processing the received sensor data by calculating the received sensor data further comprises use of a multiple linear regression algorithm having the following equation:
$$Y = {β}_{0} + {β}_{1} {X}_{1} + … {β}_{n} {X}_{n}$$
and
each of X₁ through Xₙ comprises a value taken from one or the one or more sensors.

23. (canceled)

24. (canceled)
